(19) United States
(12) Patent Application Publication
(10) Pub. No.: US 2025/0262564 A1
(43) Pub. Date: Aug. 21, 2025

(54) METHODS OF NEUTRALIZING TETRAHYDROCANNABINOL IN A CANNABINOID SAMPLE

(71) Applicant: ATLANTIC GLOBAL SUPPLY, LLC, Raleigh, NC (US)

(72) Inventor: Grania Viorela Garnighian, Colorado Springs, CO (US)

(21) Appl. No.: 19/059,163

(22) Filed: Feb. 20, 2025

Related U.S. Application Data
(60) Provisional application No. 63/555,602, filed on Feb. 20, 2024.

(51) Int. Cl. B01D 3/32; G01N 33/94

(52) U.S. Cl. CPC B01D 3/32; G01N 33/948

(57) ABSTRACT

The disclosure provides methods of reducing THC concentration of a cannabinoid sample having more than 1% THC. The methods include exposing the cannabinoid sample to an effective amount of an oxidizing agent to produce a cannabinoid product having less than 1%, e.g., less than 0.5%, less than 0.3%, or less than 0.1% THC. The methods include separating a cannabinoid product from the oxidized cannabinoid sample by phase separation and/or distillation. A system for reducing THC concentration in a cannabinoid sample is also disclosed. The system includes a reactor and a distillation column.

### CROSS-REFERENCE TO RELATED APPLICATIONS

This application claims priority under 35 U.S.C. § 119 (e) to U.S. Provisional Patent Application No. 63/555,602 titled “METHODS OF NEUTRALIZING TETRAHYDROCANNABINOL IN A CANNABINOID SAMPLE” filed Feb. 20, 2024, the entire disclosure of which is herein incorporated by reference in its entirety for all purposes.

### FIELD OF TECHNOLOGY

Aspects and embodiments disclosed herein relate generally to methods of neutralizing tetrahydrocannabinol (THC) in a cannabinoid sample, and more specifically, to methods of reducing THC concentration in a cannabinoid sample.

### SUMMARY

In accordance with one aspect, there is provided a method of reducing concentration of a psychoactive agent in a cannabinoid sample. The method may comprise obtaining the cannabinoid sample in the form of a lipid solution, the cannabinoid sample comprising a first concentration of a psychoactive agent and at least one non-psychoactive agent. The method may comprise exposing the cannabinoid sample to an effective amount of an oxidizing agent to oxidize at least a fraction of the psychoactive agent. The method may comprise combining the cannabinoid sample with a non-polar solvent to produce a miscible combination. The method may comprise separating an organic phase from the miscible combination to produce a cannabinoid product, the cannabinoid product having a second concentration of the psychoactive agent lower than the first concentration of the psychoactive agent.

In some embodiments, the second concentration of the psychoactive agent is less than 1%.

In some embodiments, the cannabinoid sample comprises or was produced from a hemp extract.

In some embodiments, the cannabinoid sample comprises or was produced from a biosynthesized composition.

In some embodiments, the method may further comprise controlling temperature and/or pressure of the cannabinoid sample.

In some embodiments, separating the organic phase from the miscible combination comprises at least one of a phase separation process and a distillation process.

In some embodiments, the psychoactive agent is tetrahydrocannabinol (THC).

In some embodiments, the THC comprises delta-8 THC, delta-9 THC, delta-10 THC, THCa, THCv, THCp, or a combination thereof.

In some embodiments, the non-psychoactive agent comprises cannabidiol (CBD), cannabinol (CBN), cannabigerol (CBG), cannabichromene (CBC), cannabidivarin (CBDV), or a combination thereof.

In some embodiments, the oxidizing agent comprises ultraviolet (UV) light, air, oxygen, ozone, peroxide, a halogen, potassium nitrate, nitric acid, or a combination thereof.

In some embodiments, the non-polar solvent comprises an alkane, an aromatic, supercritical CO₂, acetic acid, chloroform, diethyl ether, ethyl acetate, methylene chloride, pyridine, ethanol, methanol, or a combination thereof.

In some embodiments, the method further comprises obtaining a cannabinoid starting material in the form of a non-lipid starting material. The method may comprise distilling the cannabinoid starting material to obtain a cannabinoid distillate. The method may comprise combining the cannabinoid distillate with an aqueous based oil solution to obtain a cannabinoid lipid mixture. The method may comprise separating a lipid phase from the cannabinoid lipid mixture to produce the cannabinoid sample in the form of the lipid solution.

In accordance with another aspect, there is provided a method of reducing THC in a cannabinoid sample having more than 1% THC. The method may comprise exposing the cannabinoid sample to an effective amount of an oxidizing agent to oxidize a predetermined concentration of the THC and produce an intermediate product. The method may comprise measuring a concentration of THC in the intermediate product. Responsive to the concentration of THC in the intermediate product being below a predetermined threshold, the method may comprise quenching the intermediate product to produce a cannabinoid product having less than 1% THC.

In some embodiments, responsive to the concentration of THC in the intermediate product being above the predetermined threshold, the method may comprise continuing exposure of the intermediate product to the oxidizing agent.

In some embodiments, the method may further comprise separating an organic phase from the intermediate product to produce the cannabinoid product.

In some embodiments, the method may further comprise measuring the concentration of THC in the cannabinoid sample.

In some embodiments, the method may further comprise selecting the predetermined concentration of THC to be oxidized responsive to the measured concentration of THC in the cannabinoid sample.

In some embodiments, the cannabinoid product has less than 0.5% THC.

In some embodiments, the cannabinoid product has less than 0.3% THC.

In some embodiments, the cannabinoid product has less than 0.1% THC.

In accordance with another aspect, there is provided a system for reducing concentration of a psychoactive agent in a cannabinoid sample. The system may comprise a reactor having an inlet fluidly connectable to a source of the cannabinoid sample, a test sample outlet, an intermediate product outlet, and a waste outlet, the reactor comprising an insulation layer. The system may comprise a distillation column fluidly connected to the intermediate product outlet, the distillation column having a residue outlet fluidly connected to a recycle line connectable back to the distillation column and a cannabinoid product outlet. The system may comprise a temperature sensor positioned to measure temperature within the reactor. The system may comprise a pressure sensor positioned to measure pressure within the reactor. The system may comprise a source of an oxidizing agent fluidly connected to the reactor. The system may comprise a source of a non-polar solvent fluidly connected to the reactor. The system may comprise a source of a separation agent fluidly connected to the reactor. The system may comprise at least valve positioned to control flow of at least one of the oxidizing agent, the non-polar solvent, and the separation agent into the reactor.

In some embodiments, the system may further comprise a controller operably connected to the at least one valve, the controller programmed to actuate the at least one valve to control flow of the at least one of the oxidizing agent, the non-polar solvent, and the separation agent into the reactor.

In some embodiments, the system may further comprise a controller operably connected to the temperature sensor and the pressure sensor, the controller programmed to control temperature and pressure within the reactor responsive to a measurement received from the temperature sensor or the pressure sensor.

### BRIEF DESCRIPTION OF THE DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1 is a flow chart of a method of decreasing THC concentration in a cannabinoid sample, according to one embodiment

FIG. 2 is a flow chart of a method of decreasing THC concentration in a cannabinoid sample, according to one embodiment;

FIG. 3A is a box diagram of a system for decreasing THC concentration in a cannabinoid sample, according to one embodiment;

FIG. 3B is a box diagram of another system for decreasing THC concentration in a cannabinoid sample, according to one embodiment;

FIG. 4 is a box diagram of another system for decreasing THC concentration in a cannabinoid sample, according to one embodiment;

FIG. 5 is a schematic diagram of a system for decreasing THC concentration in a cannabinoid sample, according to one embodiment; and

FIG. 6 is a diagram of a chemical reaction for the conversion of CBD into THC, according to one embodiment.

### DETAILED DESCRIPTION

Federal and state regulations limit the amount of tetrahydrocannabinol (THC) that can be present in a hemp-based product. Because THC is a natural constituent of hemp extract, the amount of THC in hemp extracts and other cannabinoid biosynthesized compositions often exceeds the permissible concentration. The conventional practice to reduce THC concentration in such products is to separate THC through chromatography. However, the equipment for chromatography is complex and the process is costly. Thus, there exists a need for more efficient and flexible methods of reducing THC concentration in a cannabinoid sample.

The disclosure provides a method and system for reducing THC concentration in a cannabinoid sample. The methods disclosed herein generally involve oxidizing the cannabinoid sample under controlled conditions to reduce THC concentration while maintaining a concentration of non-psychoactive agents, such as cannabinol (CBN), above a target threshold. Thus, the cannabinoid products produced by the methods disclosed herein may have a concentration of THC that complies with federal and state regulations and a concentration of non-psychoactive agents above a target threshold.

Non-psychoactive agents in cannabinoid have been found to have beneficial properties. For example, CBN is a non-psychoactive cannabinoid agent that is a breakdown product of THC. CBN is typically found in trace amounts in a cannabinoid sample. Recent studies have found certain therapeutic benefits of CBN, and interest in this minor cannabinoid agent is increasing.

While conventional methods of reducing THC concentration typically involve separation of THC, the methods disclosed herein instead use controlled conditions to convert THC into CBN, providing the added benefit of increasing CBN concentration in the cannabinoid product. Furthermore, the inventors surprisingly discovered that controlling certain process conditions during the oxidation of THC, such as temperature, light, heat, and/or pressure, may provide unexpected efficiencies in the conversion of THC to CBN, producing a superior product.

The cannabinoid starting material utilized in the methods disclosed herein may comprise a hemp extract. As used herein, the hemp extract may comprise a hemp plant (family Cannabaceae plants, e.g., Cannabis sativa) extract from leaves, flowers, stems, seeds, or a combination thereof. Thus, the hemp extract may be whole plant (full or broad spectrum) extract, hemp seed oil, hemp chaff, a cannabinoid isolate or oil, e.g., CBD isolate or oil, or a combination thereof. The hemp extract may comprise a constituent of a hemp extract, e.g., an isolated or purified constituent of a hemp extract.

In certain embodiments, the cannabinoid starting material may comprise a biosynthesized composition. Biosynthesized compositions are typically formed from a cannabinoid precursor compound reacted with a catalyst under favorable conditions to produce the biosynthesized composition. In some embodiments, the cannabinoid precursor compound may itself be a hemp extract or constituent of a hemp extract. In other embodiments, the cannabinoid precursor compound may be synthesized. In some embodiments, the cannabinoid starting material may comprise both a hemp extract and a biosynthesized composition. For instance, in certain embodiments, a hemp extract may be fortified with a biosynthesized composition.

The cannabinoid may comprise psychoactive and non-psychoactive agents. The psychoactive agents may include THC, e.g., one or more of delta-8 THC, delta-9 THC, delta-10 THC, or other isomers, THCa, THCv, and/or THCp. The non-psychoactive agents may include one or more of cannabidiol (CBD), cannabinol (CBN), cannabigerol (CBG), cannabichromene (CBC), cannabidivarin (CBDV), and others. The methods disclosed herein may be controlled to reduce concentration of psychoactive agents, e.g., THC, to below a target threshold, while substantially maintaining a concentration of non-psychoactive agents above a target threshold. In certain embodiments, the concentration of non-psychoactive agents may be increased from the concentration of non-psychoactive agents in the cannabinoid. For example, the methods disclosed herein may be controlled to increase CBN concentration to a value that is above a greater target threshold.

Thus, in accordance with one aspect, there is provided a method of reducing THC concentration in a cannabinoid sample. The cannabinoid sample may be in the form of a lipid-based solution, e.g., an oil. The methods may comprise providing the cannabinoid sample as a lipid-based solution.

In other embodiments, the methods may comprise preparing a lipid-based cannabinoid sample, e.g., an oil, from a cannabinoid starting material, such as a hemp extract or non-lipid starting material. Cannabinoid oil may be produced by any method known in the art. In certain exemplary embodiments, the methods may comprise distilling a cannabinoid starting material to obtain a cannabinoid distillate, combining the cannabinoid distillate with an aqueous based oil solution, and separating the lipid phase.

In some embodiments, the cannabinoid distillate may be produced by a molecular distillation process, similar to the process of distilling alcohol. The cannabinoid starting material may be heated to the point of vaporization or to a temperature effective to cause decarboxylation (activation) of THC, producing an evaporated cannabinoid material. The evaporated cannabinoid material may be decondensed to produce a concentrated distillate and, optionally, collected.

To produce the cannabinoid sample, the concentrated distillate may be combined in an aqueous solution having an effective concentration of oil to produce an aqueous cannabinoid solution. In certain embodiments, the effective concentration of oil may be 5-30% oil, e.g., 5-10% oil, 10-20% oil, or 20-30% oil. The aqueous cannabinoid solution may be heated to an effective temperature at an effective pressure for lipid separation. In certain exemplary embodiments, the aqueous solution may be heated to a temperature of 120° C. at a pressure greater than 1 atm. The cannabinoid aqueous solution may be cooled to separate a lipid part from an aqueous part. The lipid part may be collected and solidified. The separation process (e.g., heating, cooling, and separation) may be repeated as necessary to obtain the cannabinoid sample having desired properties, e.g., a desired purity. In certain embodiments, the cannabinoid sample may then be heated to an effective temperature to vaporize any residual water. An exemplary vaporization temperature is about 140° C.

The cannabinoid sample may have a THC concentration that exceeds the regulated amount, for example, exceeds 0.3% THC. In some embodiments, the cannabinoid sample may have a THC concentration of 1-10% or more. The methods disclosed herein may be utilized to reduce the THC concentration by 70-90% or more, e.g., 70-99%. In some embodiments, the methods disclosed herein may be utilized to produce a cannabinoid product having 0.01-1% THC, for example, 0.01-0.5%, 0.01-0.3%, 0.01-0.2%, 0.01-0.1%, or less. In some embodiments, the cannabinoid product may have an undetectable amount of THC, for example, the concentration of THC may be below a detection limit of the appropriate detection instrument. The methods may comprise exposing the cannabinoid sample to an oxidant to produce an oxidized composition. The oxidant may be introduced in an amount effective to oxidize a pre-determined concentration of the THC present in the sample.

Under oxidation conditions, cannabinoid compounds either lose hydrogen atoms or gain oxygen atoms. THC (C₂₁H₃₀O₂) is converted to CBN (C₂₁H₂₆O₂). Thus, oxidation may produce a cannabinoid product having reduced THC and increased CBN. Accordingly, under oxidation conditions, not only may psychoactive properties of the sample be reduced, but beneficial non-psychoactive properties may be increased. Furthermore, CBN is believed to counteract unwanted side effects of THC consumption.

However, under oxidation conditions, CBD is also oxidized, becoming hydroxyquinone. Thus, in order to preserve the concentration of non-psychoactive agents in the sample, the oxidation conditions may be controlled, (for example, by controlling exposure time to the oxidizing agent or concentration or intensity of the oxidizing agent introduced), to limit oxidation of non-psychoactive agents, such as CBD. Accordingly, the effective amount of the oxidizing agent may be selected to oxidize a pre-determined amount of the THC in the cannabinoid sample. The pre-determined amount of the THC to be oxidized may be enough to reduce the THC concentration to less than 1%, for example, less than 0.5%, less than 0.3%, less than 0.1%, or less than 0.01%, while maintaining a concentration of non-psychoactive agents above a target threshold.

Oxidation may be performed by exposing the sample to an effective amount of one or more oxidizing agents. Oxidizing agents may include, for example, ultraviolet (UV) light, air, oxygen, ozone, peroxide, halogens, potassium nitrate, nitric acid, and others, or combinations thereof. In some embodiments, the cannabinoid sample may be exposed to two or more oxidizing agents simultaneously or sequentially.

The amount or rate of oxidation may be controlled by exposing the cannabinoid sample to the oxidizing agent for a selected amount of time. For example, the sample may be exposed to the oxidizing agent for 12-72 hours, for example, 12-24 hours, 24-48 hours, or 48-72 hours. In some embodiments, the amount or rate of oxidation may further be controlled by selecting a concentration or intensity of the oxidizing agent introduced. The oxidizing agent may be introduced at a concentration of 25-75%, for example, at least 25%, about 25%, 25-50%, at least 50%, about 50%, 50-75%, or about 75%.

In some embodiments, the effective amount of the oxidizing agent (e.g., exposure time and/or concentration of the oxidizing agent) may be selected based on the concentration of THC in the cannabinoid sample. Thus, in some embodiments, the methods may comprise measuring THC concentration in the cannabinoid sample. In certain exemplary embodiments, THC concentration may be measured by high-performance liquid chromatography (HPLC). In certain embodiments, THC concentration may be measured by gas chromatography (GC). Other methods of measuring THC concentration may also be used. The effective amount of the oxidizing agent may be selected responsive to the measured THC concentration.

The methods may include monitoring the cannabinoid sample to determine the rate of oxidation. Degradation of THC may be monitored based on time of exposure to the oxidizing agent. In some embodiments, the cannabinoid sample may be monitored periodically. For instance, the methods may comprise collecting a sample of the composition and testing the sample. One or more property of the sample may be tested, for example, THC concentration (which may be measured by HPLC, GC, or other method), non-psychoactive agent concentration (e.g., CBD and/or CBN concentration), pH, temperature, etc. The methods may comprise testing the sample every 6-24 hours, for example, every 6 hours, every 12 hours, every 18 hours, or every 24 hours. In other embodiments, the cannabinoid sample may be monitored substantially continuously. For instance, one or more sensor may be positioned to monitor a property of the cannabinoid sample, such as composition, temperature, pH, etc.

Degradation of THC by oxidation may be allowed to continue until the THC concentration is below a predetermined threshold. In some embodiments, the predetermined threshold may be a regulatory limit, for example, a federal or state regulatory concentration. In some embodiments, the predetermined threshold THC concentration is 1%, for example, 0.5%, 0.3%, 0.1%, or 0.01%. In some embodiments, the predetermined threshold THC concentration is a detection limit. Thus, in certain embodiments, the degradation of THC by oxidation may be allowed to continue until THC concentration is undetectable in the sample. The methods may include stopping or quenching the oxidation reaction responsive to measuring or detecting a THC concentration in the cannabinoid sample below the predetermined threshold.

In some embodiments, the methods may comprise controlling temperature and/or pressure during the oxidation reaction. Without wishing to be bound by theory, it is believed that heating the reaction may further increase the rate of oxidation. The reaction may be heated to a temperature above 25° C., for example, 30-60° C., e.g., 30-40° C., 40-60° C., or more. Temperature of the cannabinoid sample during oxidation may be periodically or continuously monitored. In some embodiments, the reaction may be performed at an increased pressure, for example, a pressure above 1 atm, for example, 1-4 atm, e.g., 1-2 atm, 2-3 atm, 3-4 atm, or more. Pressure of the oxidation reaction, for example, pressure within a reactor, may be periodically or continuously monitored.

The methods may comprise combining the cannabinoid sample or oxidized composition with an effective amount of a non-polar solvent to produce a miscible combination. The non-polar solvent may be introduced before the oxidizing agent, simultaneously with the oxidizing agent, or after the oxidizing agent. The lipid-based cannabinoid solution is non-polar and will dissolve in the non-polar solvent. While not wishing to be bound by theory, it is believed that dissolving the cannabinoid sample in a non-polar solvent has a synergistic effect with oxidation. For instance, the non-polar solvent may be effective at increasing exposure of THC molecules in the cannabinoid oil to the oxidizing agent.

Thus, in certain embodiments, the cannabinoid sample may be combined with a non-polar solvent to produce the miscible combination prior to oxidation or prior to the completion of the oxidation. The miscible combination may then be exposed to an effective amount of an oxidizing agent, as previously described to produce an oxidized combination. However, in other embodiments, the oxidized composition may be combined with the non-polar solvent to produce the miscible combination after oxidation.

Exemplary non-polar solvents include alkanes (pentane, hexane, heptane, propane, and butane), aromatics (benzene, toluene, and xylene), supercritical CO₂, acetic acid, chloroform, diethyl ether, ethyl acetate, methylene chloride, pyridine, ethanol, methanol, and combinations thereof. The effective amount of the non-polar solvent may be an amount effective to dissolve the terpenes and cannabinoids in the cannabinoid sample.

The reaction may be allowed to proceed for a pre-determined amount of time before quenching by addition of purified water. In some embodiments, the reaction may be allowed to proceed for 6-24 hours, for example, 6-12 hours, 12-18 hours, or 18-24 hours.

The methods disclosed herein may further comprise separating a cannabinoid product from the combination. In some embodiments, the separation may include a phase separation of an organic phase (product) from an aqueous phase. The composition may be combined with an effective amount of a separation agent and allowed to settle into the separate phases. The combination may be allowed to settle until a clean line of separation is observed. In some embodiments, the combination may be allowed to settle for at least 15 minutes, for example, 15-30 minutes, 30-40 minutes, 40-50 minutes, 50-60 minutes, or more.

Exemplary separation agents include salts, such as sodium bicarbonate (NaHCO₃), sodium chloride (NaCl), sodium sulfate (Na₂SO₄), and hydrates thereof. The separation agent may be highly soluble in water. Since the organic phase is separated from an aqueous phase, the separation agent may be selected to dissolve and eliminate an effective amount of water-soluble contaminants.

After the phase separation, the organic phase may be collected as the cannabinoid product, and the aqueous phase may be discarded. In certain embodiments, the phase separation may be performed one or more times in series. For instance, an effective amount of the separation agent may be added to the organic phase and allowed to settle. Additional aqueous phase may be separated and discarded. The separation may be performed as many times as necessary to achieve a desired purity of the cannabinoid product. In some embodiments, the separation may be performed 1, 2, 3, 4, 5, or more times. In some embodiments, the collected organic phase (product) may be filtered to remove any solid or precipitated contaminants. The organic phase may be subjected to evaporation to produce a dry cannabinoid product.

The separation may additionally or alternatively include a distillation of the composition. Distillation may be performed to remove any residual contaminant, e.g., non-polar solvent, from the composition. After distillation, the distillate may be collected as the cannabinoid product, and the residue may be discarded.

The distillation may be performed one or more times in series. For instance, the distillate may be subjected to a further distillation. The distillation may be performed as many times as necessary to achieve a desired purity of the cannabinoid product. The distillation may be performed until the cannabinoid product is below a target concentration of contaminants, e.g., non-polar solvent. In certain embodiments, the distillation may be performed until the concentration of non-polar solvent is below a detection threshold in the cannabinoid product. In some embodiments, the distillate may be filtered to remove any solid or precipitated contaminants. The distillate may be subjected to evaporation to produce a dry cannabinoid product.

In some embodiments, the cannabinoid product may be produced by phase separation. In some embodiments, the cannabinoid product may be produced by distillation. In other embodiments, the cannabinoid product may be produced by phase separation and distillation. One or more distillation procedure may follow one or more phase separation procedure. Alternatively, one or more phase separation procedure may follow one or more distillation procedure. The cannabinoid product may have a THC concentration below a target threshold, a non-psychoactive agent concentration above a target threshold, and a desired purity (e.g., non-detectable residual non-polar solvent).

The methods may comprise measuring THC concentration and/or non-psychoactive agent concentration in the cannabinoid product or an intermediate composition. For example, in some embodiments, THC concentration of an intermediate composition may be measured before continuing to a further step in the process. One or more steps may be repeated until a desired THC concentration is achieved. Thus, in some embodiments, THC and/or non-psychoactive agent concentration may be measured after oxidation. In some embodiments, THC and/or non-psychoactive agent concentration may be measured after combination with the non-polar solvent. In some embodiments, THC and/or non-psychoactive agent concentration may be measured in the cannabinoid product. THC and non-psychoactive agent, e.g., CBD and/or CBN, concentration may be measured by HPLC, GC, and/or other methods.

The cannabinoid product may be distributed as a raw material or incorporated in a finished product. For instance, in certain embodiments, the cannabinoid product may be formulated as or incorporated into any natural product, dietary supplement, or consumer product. Examples of finished products include oils, foods, drinks, nutraceuticals or supplements, capsules, topical compositions, such as lotions, creams, gels, aerosols, sprays, mists, serums, and salves, wearable articles, such as patches, masks, bandages, and dressings, and cosmetic compositions, such as soaps, shampoos, conditioners, moisturizers, cleansers, and fragrances.

An effective amount of the cannabinoid product may be incorporated in the finished product. In some embodiments, the finished product may comprise 0.001%-99.999% of the cannabinoid product, e.g., 0.001-0.01%, 0.01-0.1%, 0.1-1%, 1-5%, 5-10%, 10-20%, 20-30%, 30-40%, 40-50%, 50-60%, 60-70%, 70-80%, 80-90%, 90-95%, 95-99%, 99-99.9%, 99.9-99.99%, or 99.99-99.999%. The finished product may further comprise one or more excipient suitable for the formulation of the finished product. Exemplary excipients include thickeners, surfactants, rheology modifiers, humectants, emollients, preservatives, and others. In some embodiments, the cannabinoid product or formulation may be coated on, embedded in, or otherwise provided with a substrate, for example, to produce a wearable article.

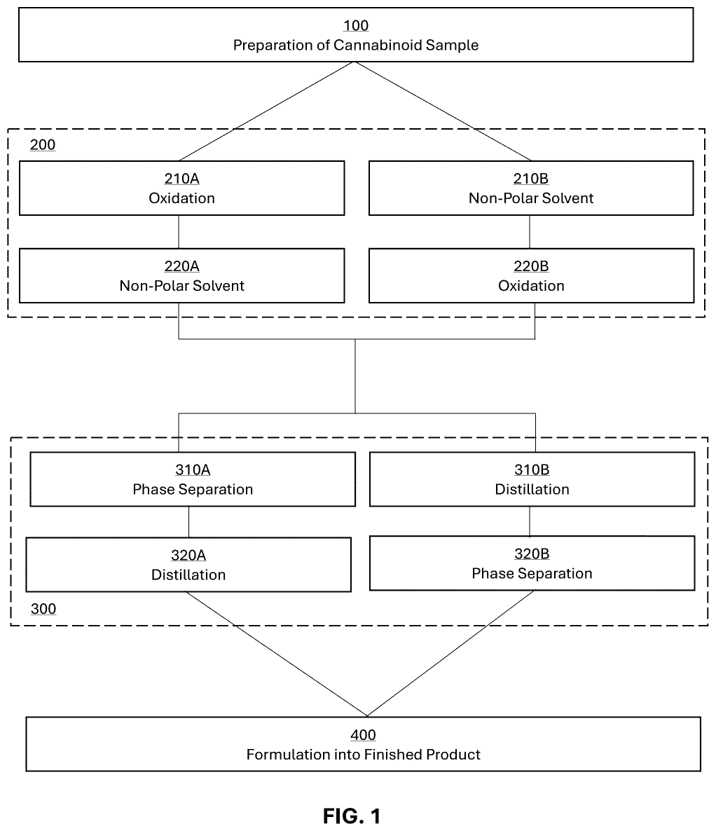

FIG. 1 is a flow chart of one exemplary method of decreasing THC concentration in a cannabinoid sample. As shown in FIG. 1, a cannabinoid sample may be prepared (100). The cannabinoid sample may be treated for reduction of THC concentration (200). THC neutralization (200) may include exposure to an oxidant (210A) and a non-polar solvent (220A). Optionally, the THC neutralization (200) steps may be reversed by exposing the sample to a non-polar solvent (210B) and an oxidant (220B). In yet other embodiments, THC neutralization (200) may include exposing the sample to the oxidant and non-polar solvent substantially simultaneously. In certain embodiments, the sample may be exposed to the non-polar solvent before completion of the oxidation reaction.

The oxidized sample may be separated (300) to purify a cannabinoid product. The separation process (300) may include a phase separation (310A) and/or a distillation (320A). In some embodiments, the cannabinoid product may be treated by phase separation (310A) followed by distillation (320A). In other embodiments, the separation process (300) may be performed as a distillation (310B) followed by a phase separation (320B). The product obtained from the separation may be distributed as a raw material or formulated into a finished product (400).

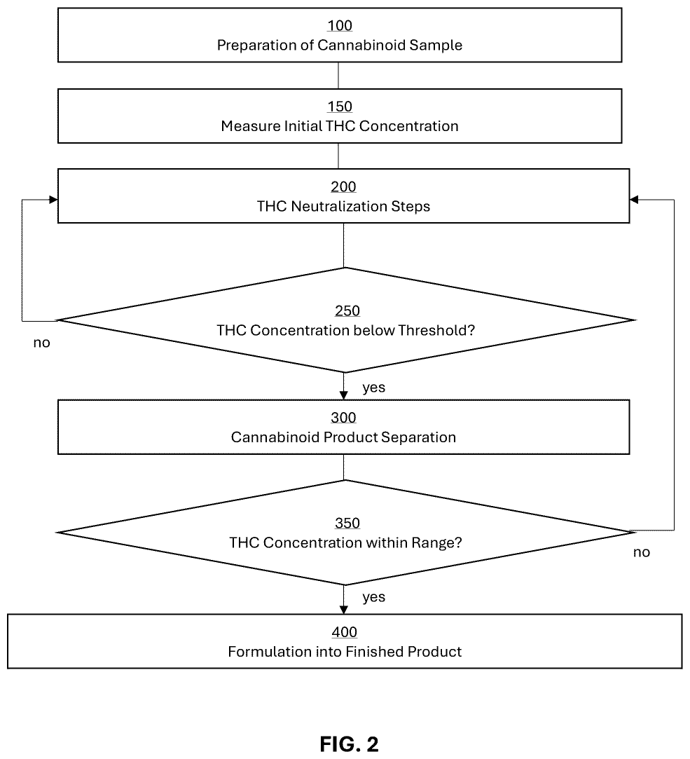

FIG. 2 is a flow chart of one exemplary method of decreasing THC concentration in a cannabinoid sample. The exemplary method of FIG. 2 includes THC concentration measurement steps. As shown in the exemplary flow chart of FIG. 2, in some embodiments, an initial THC concentration measurement (150) may be taken from the prepared cannabinoid sample (100). Parameters for the THC neutralization steps (200) may be selected responsive to the initial THC concentration measurement (150). THC concentration may be measured (250) after THC neutralization (200). Responsive to the THC concentration being below a target threshold, the cannabinoid product may be separated (300) from the oxidized sample. Responsive to the THC concentration not being below a target threshold, THC neutralization steps (200) may be repeated or continued.

After separation of the cannabinoid product (300), THC concentration may be measured (350). Responsive to the THC concentration being within a target range (e.g., below a target threshold), the product may be approved and, optionally, distributed as a raw material or formulated into a finished product (400). Responsive to the THC concentration not being within a target range (e.g., not being below a target threshold), the product may be treated as a cannabinoid sample, and THC neutralization steps (200) may be repeated.

In some embodiments, concentration of a non-psychoactive agent, such as CBD and/or CBN, may be measured in addition to or instead of THC concentration. The process may be similar to the flow chart shown in FIG. 2. For instance, THC neutralization steps (200) may be repeated responsive to the non-psychoactive agent concentration being outside a target range. In certain embodiments, for example, responsive to CBD concentration being below a target threshold, the THC neutralization steps (200) may be stopped. In such embodiments, if THC is above a target threshold, other methods of THC removal or neutralization may be performed, to avoid further degradation of THC.

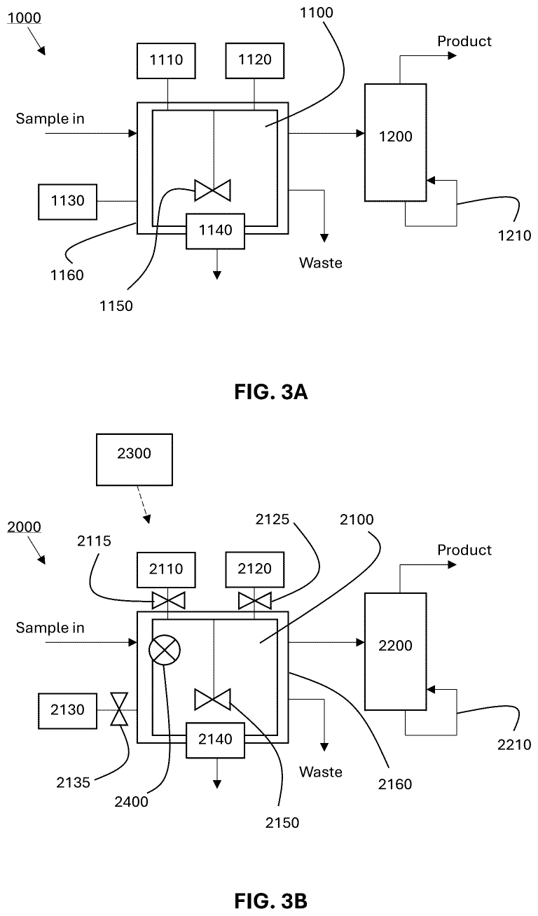

In accordance with one aspect, there is provided a system for reduction of THC concentration in a sample. FIG. 3A is a box diagram of an exemplary system 1000. System 1000 includes reactor 1100, which has a sample inlet, a waste outlet, and an intermediate product outlet. Distillation column 1200 is fluidly connected to the reactor 1100 via the intermediate product outlet. Distillation column 1200 includes a distillate outlet, a residue outlet, and optional residue recycle line 1210. The system 1000 includes a source of an oxidizing agent 1110, a source of a non-polar solvent 1120, and a source of a separation agent 1130, each fluidly connected to the reactor 1100. The reactor 1100 includes a sample port 1140 positioned to allow collection of a test sample of the cannabinoid substance within the reactor 1100. The reactor 1100 includes a stirrer 1150 positioned to stir the cannabinoid substance within the reactor 1100. The reactor 1100 includes an insulation layer 1160 positioned to maintain temperature control of the cannabinoid substance within the reactor 1100.

It should be noted that even though exemplary system 1000 shows a single reactor 1100, in some embodiments, the reactor 1100 may be provided as two or more reactors. For instance, in some embodiments, the system may include an oxidation reactor fluidly connected to a source of an oxidant and a source of a non-polar solvent. The system may include a separator fluidly connected to an intermediate product outlet of the oxidation reactor. The separator may have a waste outlet and an intermediate product outlet. The distillation column may be fluidly connected to the intermediate product outlet of the separator. Thus, reactor 1100 may be provided as two or more reactors in series.

Furthermore, it should be noted that while exemplary system 1000 shows a single distillation column 1200, in certain embodiments, the distillation column 1200 may be provided as two or more distillation columns. A first distillation column may be fluidly connected to an intermediate product outlet of the reactor. A second distillation column may be fluidly connected to a residue outlet of the first distillation column. In such embodiments, two-pass distillation may be performed in separate distillation columns.

In certain embodiments, system 1000 may include two or more reactors and/or two or more distillation columns provided in parallel. Parallel unit operations may be used to scale up the cannabinoid sample processing methods described herein.

FIG. 3B is a box diagram of another exemplary system 2000 for reduction of THC concentration in a sample. The system 2000 of FIG. 3B is similar to system 1000 of FIG. 3A. System 2000 includes a reactor 2100 having a sample inlet, a waste outlet, and an intermediate product outlet and distillation column 2200 fluidly connected to the reactor 2100 via the intermediate product outlet. Distillation column 2200 includes a distillate outlet, a residue outlet, and optional residue recycle line 2210. The reactor 2100 includes a sample port 2140, a stirrer 2150, and an insulation layer 2160. Similar to system 1000, in some embodiments, reactor 2100 may be provided as two or more reactors and/or distillation column 2200 may be provided as two or more distillation columns.

System 2000 includes a source of an oxidizing agent 2110, a source of a non-polar solvent 2120, and a source of a separation agent 2130, each fluidly connected to the reactor 2100. Pumps and/or valves 2115, 2125, 2135 are positioned to control flow of the oxidizing agent 2110, non-polar solvent 2120, and separation agent 2130, respectively, into the reactor 2100. Exemplary system 2000 includes optional controller 2300 operably connected to one or more of pumps and/or valves 2115, 2125, 2135. The controller 2300 may be programmed to actuate one of more of pumps and/or valves 2115, 2125, 2135 to control flow of the oxidizing agent 2110, non-polar solvent 2120, and separation agent 2130, respectively, in accordance with a protocol. In some embodiments, the controller 2300 may be programmed to control one or more parameter within reactor 2100, such as temperature, pressure, and/or pH of the cannabinoid substance.

In some embodiments, controller 2300 may be manually activated. For instance, the controller 2300 may actuate one or more of pumps and/or valves 2115, 2125, 2135 responsive to an instruction provided by the user. In some embodiments, controller 2300 may be automatically activated. For instance, the controller 2300 may actuate one or more of pumps and/or valves 2115, 2125, 2135 responsive to instructions encoding a protocol. Optionally, controller 2300 may actuate one or more of pumps and/or valves 2115, 2125, 2135 responsive to a measurement obtained from at least one sensor 2400.

System 2000 may comprise at least one sensor 2400. Sensor 2400 may be positioned to measure at least one parameter within the reactor 2100. Exemplary sensors 2400 include composition sensors, temperature sensors, pressure sensors, and pH sensors, among others. In some embodiments, the sensor 2400 may be connectable to a network and programmed to communicate a measurement to a user. For instance, the sensor 2400 may be programmed to communicate a measurement to a computing or mobile device operated by the user. Optionally, the sensor 2400 may be programmed to alert a user of a measurement that is outside of a threshold range.

In certain embodiments, the sensor 2400 may be operably connectable to the controller 2300. The controller 2300 may optionally be programmed to adjust a parameter of the reactor 2100 responsive to a measurement obtained by the sensor 2400. For instance, the controller 2300 may be programmed to adjust temperature, pressure, or pH within the reactor 2100 responsive to a measurement obtained by the sensor 2400. In certain embodiments, the controller 2300 may be programmed to actuate one or more of pumps and/or valves 2115, 2125, 2135 to control flow of the oxidizing agent 2110, non-polar solvent 2120, and separation agent 2130, respectively, responsive to a measurement obtained by the sensor 2400.

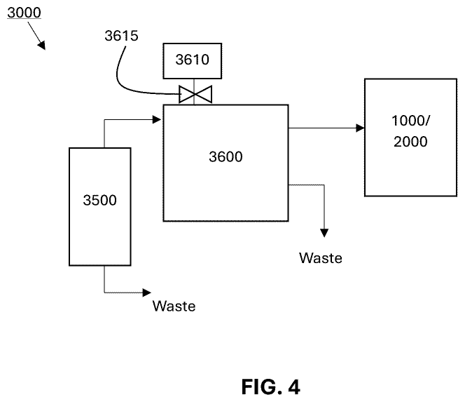

FIG. 4 is a box diagram of another system 3000 for reduction of THC in a cannabinoid sample. System 3000 includes distillation column 3500 and reactor 3600 for production of a cannabinoid oil from a starting material. Distillation column 3500 includes an inlet for the starting material, a distillate outlet, and a residue outlet. Reactor 3600 is fluidly connected to distillation column 3500 via the distillate outlet. While not shown in the exemplary embodiment of FIG. 4, in some embodiments, distillation column 3500 may include a residue recycle line similar to recycle lines 1210, 2210.

System 3000 includes a source of an aqueous solution 3610 fluidly connected to reactor 3600 and pump or valve 3615 positioned to control flow of the aqueous solution into the reactor 3600. Reactor 3600 has cannabinoid sample outlet and a waste outlet. The cannabinoid sample outlet is fluidly connected to a sample inlet of a system for reduction of THC, such as systems 1000, 2000. While not shown in the exemplary embodiment of FIG. 4, reactor 3600 may include one or more of a stirrer, a sample port, and/or an insulation layer. Furthermore, in some embodiments, pump and/or valve 3615 may be operably connected to a controller programmed to actuate pump and/or valve 3615 to control flow of the aqueous solution, such as controller 2300. Controller 2300 or another controller may be programmed to control a parameter within reactor 3600, such as temperature, pressure, and/or pH of the cannabinoid substance.

Similar to distillation columns 1200, 2200 and reactors 1100, 2100, in some embodiments, distillation column 3500 and reactor 3600 may be provided as two or more distillation columns and/or reactors. In some embodiments, the two or more distillations and/or reactors may be fluidly connected in series. In some embodiments, the two or more distillation columns and/or reactors may be arranged in parallel. Furthermore, in some embodiments, reactor 3600 and one of reactors 1100, 2100 may be a single reactor. For instance, distillation column 3500 may be fluidly connected directly to the sample inlet of reactor 1100, 2100. The source of the aqueous solution 3610 may be fluidly connected directly to reactor 1100, 2100.

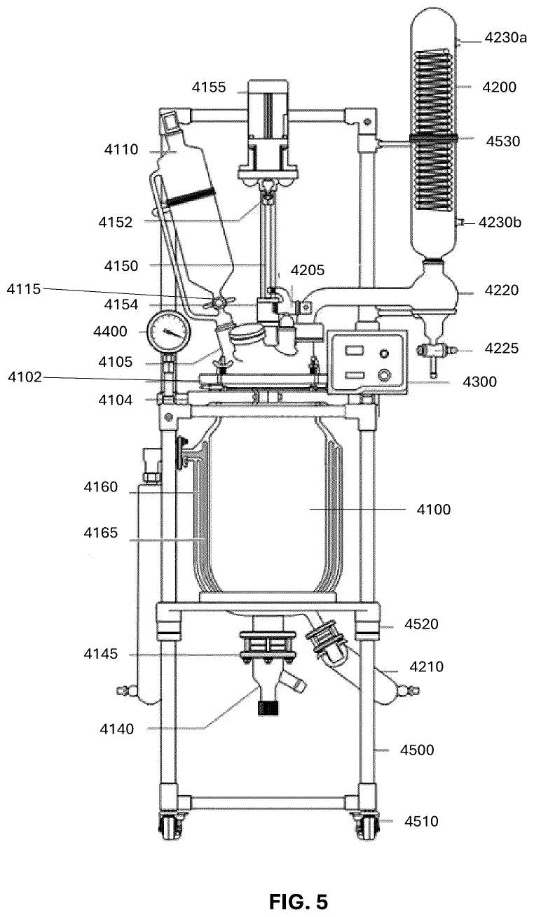

FIG. 5 is a schematic diagram of an exemplary system for reduction of THC concentration in a sample. The system of FIG. 5 includes a reactor 4210 having an outer vacuum jacket 4160 and an inner cold/heat recirculating fluid jacket 4165 for temperature control. The reactor 4100 has a sample port 4140 with sealed aluminum flanges 4145 and a glass drain port having a PTFE valve. The reactor 4100 includes an insulated recirculating fluid connection hose 4210 that is connectable back to the reactor 4100. The system includes a feeding tunnel 4110 fluidly connected to an upper chamber of the reactor 4100 through a reaction vessel lid 4105. The feeding tunnel 4110 may be used to introduce the sample, the oxidizing agent, the non-polar solvent, the separation agent, and/or an aqueous solution into the reactor 4100. A valve 4115 is positioned to allow flow of the solution within the feeding tunnel 4110 into the reactor 4100.

A stirrer 4150 is positioned to stir reagents introduced through the feeding tunnel 4110. The stirrer 4150 is operably connected to a motor 4155 configured to actuate the stirrer 4150. The stirrer 4150 is fixed between a universal joint 4152 and a stirrer bearing 4154. An upper sealing clamp 4102 and lower sealing clamp 4104 fix the reactor 4100 to a stand 4500. The stand 4500 includes wheels 4510 on a swivel with a lock, making the system mobile. The stand 4500 also includes an adjusting nut 4520 that may be used to increase or decrease height of the system.

A condenser 4200 is fluidly connected to the reactor 4100 through a glass vacuum valve 4205. The condenser 4200 is attached to the stand 4500 by a rubber bracket 4530. The condenser 4200 includes a condenser dispenser 4220 and a glass drain port 4225 through which the distillate may be removed as the cannabinoid product. A cooling fluid may be introduced into the condenser through chilling fluid ports 4230a, 4230b. The exemplary system of FIG. 5 is capable of performing distillation between the reactor 4100 and condenser 4200.

The system of FIG. 5 includes a control panel 4300 that displays temperature within the reactor 4100 and rotation speed of the stirrer 4150. The temperature measurement may be provided to the control panel 4300 by a temperature sensor or as a known temperature of the cold/hot recirculating fluid. The stirrer speed may be provided to the control panel 4300 by the motor 4155. In certain embodiments, the motor 4155 is operably connected to the control panel 4300, and stirrer speed may be controlled though the control panel 4300. The system of FIG. 5 also includes a mechanical vacuum gauge 4400 positioned to measure and display pressure within the reactor 4100.

### Examples

The function and advantages of these and other embodiments can be better understood from the following examples. These examples are intended to be illustrative in nature and are not considered to be limiting the scope of the invention.

### Example 1: Preparation of a Lipid-Based Cannabinoid Sample

The lipid-based cannabinoid sample may be prepared from a hemp extract or biosynthesized composition. A concentrated distillate of the hemp extract or biosynthesized composition may be obtained. The concentrated distillate may be combined with an aqueous oil composition. A lipid-based sample having the cannabinoid may then be separated from the aqueous oil composition.

To prepare the concentrated distillate, the extract or biosynthesized composition is heated to the point of vaporization, which causes decarboxylation (activation) of THC. Then the evaporated material is decondensed and collected as the concentrated distillate.

To prepare the lipid-based cannabinoid sample from the concentrated distillate, the concentrated distillate is combined with an aqueous solution having a concentration of 10-20% oil. The solution is heated to 120° C. at a pressure greater than 1 atm. The solution is cooled to separate lipids having the cannabinoid from the aqueous part and the lipid part is solidified. The process is repeated as necessary to obtain a lipid-based cannabinoid sample having the desired properties, e.g., a desired purity. The lipid-based cannabinoid sample is then heated to 140° C. to vaporize any residual water.

### Example 2: Reduction of THC Concentration

The cannabinoid sample obtained in Example 1 is then oxidized by exposure to ozone and UV light for approximately 12-48 hours. The sample is dosed at a concentration of about 50% oxidizing agent.

A heptane solution is added to the cannabinoid sample, before or during oxidation, to produce a miscible combination. The mixture is heated to 40° C. and allowed to proceed for approximately 12 hours. At this stage, THC concentration is measured by HPLC and GC to confirm concentration. If the THC concentration is above a predetermined threshold, the oxidation reaction is allowed to continue. THC concentration may be measured every 12 hours.

Once the concentration is confirmed to be below the predetermined threshold, e.g., 0.3% THC, the reaction is quenched by addition of reverse osmosis (RO) purified water.

### Example 3: Separation of the Cannabinoid Product from the Oxidized Composition

The composition from Example 3 is stirred vigorously for at least 5 minutes. The composition is then allowed to settle for at least 15 minutes, or a sufficient amount of time to separate the organic and aqueous phases. The organic phase is collected and the aqueous phase is then discarded.

The organic phase is then saturated with a sodium bicarbonate solution and stirred until an aqueous phase forms. If necessary, the composition may be allowed to settle for at least 15 minutes, or a sufficient amount of time to separate the aqueous phase. The organic phase is collected and the aqueous phase is again discarded.

The organic phase is then mixed with a sodium chloride solution and stirred vigorously for at least 5 minutes, then allowed to settle for at least 15 minutes, or a sufficient amount of time to separate the aqueous phase. The organic phase is again collected and the aqueous phase is again discarded.

The remaining organic portion is combined with sodium sulfate and allowed to dry for at least 30 minutes. The mixture is filtered with a cellulose filter and evaporated in a round bottom flask to produce a dry reaction crude sample.

The cannabinoid product in the crude sample is then separated by a two-pass distillation. After the first pass distillation, the residue is collected as distilland. After the second pass distillation, the distillate is collected as the cannabinoid product. The residue is separately collected.

The cannabinoid product from the distillate is then tested by HPLC to confirm that the THC concentration is below a target threshold.

### Example 4: Conversion of CBD into THCa

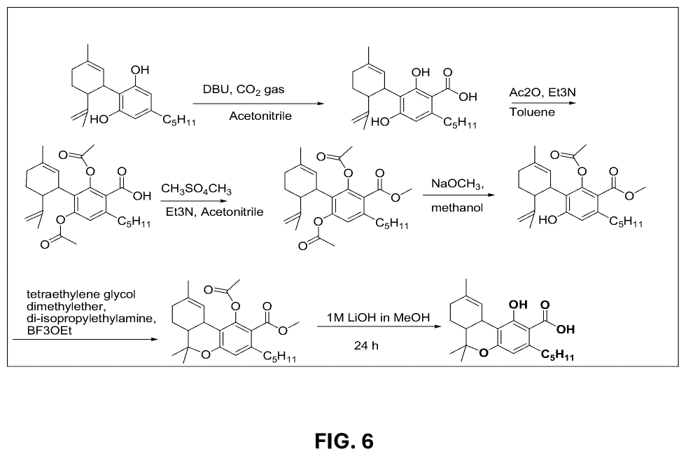

In accordance with the methods disclosed herein, CBD may be converted into THC by a reaction summarized in the diagram of FIG. 6. Briefly, CBD may be carboxylated by reaction with 1,8-diazabicyclo [5.4.0]undec-7-ene (DBU), CO₂ gas, and acetonitrile. The resulting compound may be acetylated by reaction with acetic anhydride (Ac2O), triethyl amine (Et3N), and dimethylformamide (DMF) or toluene. The resulting compound may be esterified in a two step process by reaction with dimethyl sulfate (CH₃SO₄CH₃), triethylamine (Et₃N), and acetonitrile, and then methyl iodide, NaHCO₃, and DMF. The resulting compound may be deacetylated by reacting with sodium methanolate (sodium methoxide, NaOCH₃) or 1M solution of ammonia in methanol and purified with ion exchange resin (Amberlite IR-120 (H), distributed by Sigma-Aldrich, St. Louis, MO). The resulting CBDA-acetate methyl ester compound may be cyclized by reaction with tetra ethylene glycol dimethyl ether, di-isopropyl ethylamine, boron trifluoride diethyl etherate (BF3OEt), and dichloromethane (DCM), becoming THCa-acetate methyl ester.

The THCa-acetate methyl ester may be converted into THCa by hydrolysis in a reaction with 1 M lithium hydroxide (LiOH) in methanol (MeOH) solution. The hydrolysis of methyl ester of cannabinoids is a two-step process: in part A of the first step, the composition is combined with CsCO₃ (1.2 equivalents) in MeOCH₂CH₂OMe, at 80° C. for 1 h. In part B of the first step, the composition is combined with CsCO₃ (2.5 equivalents) in MeOH for 2 h. In the second step, the composition is combined with KOH (1 equivalent) in a water/ethanol (EtOH)(1:1) mixture and refluxed for 30 minutes.

With the process described above, CBD may be converted to THCa.

The phraseology and terminology used herein is for the purpose of description and should not be regarded as limiting. As used herein, the term “plurality” refers to two or more items or components. The terms “comprising,” “including,” “carrying,” “having,” “containing,” and “involving,” whether in the written description or the claims and the like, are open-ended terms, i.e., to mean “including but not limited to.” Thus, the use of such terms is meant to encompass the items listed thereafter, and equivalents thereof, as well as additional items. Only the transitional phrases “consisting of” and “consisting essentially of,” are closed or semi-closed transitional phrases, respectively, with respect to the claims. Use of ordinal terms such as “first,” “second,” “third,” and the like in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Having thus described several aspects of at least one embodiment, it is to be appreciated various alterations, modifications, and improvements will readily occur to those skilled in the art. Any feature described in any embodiment may be included in or substituted for any feature of any other embodiment. Such alterations, modifications, and improvements are intended to be part of this disclosure and are intended to be within the scope of the invention. Accordingly, the foregoing description and drawings are by way of example only.

Those skilled in the art should appreciate that the parameters and configurations described herein are exemplary and that actual parameters and/or configurations will depend on the specific application in which the disclosed methods and materials are used. Those skilled in the art should also recognize or be able to ascertain, using no more than routine experimentation, equivalents to the specific embodiments disclosed.

1. A method of reducing concentration of a psychoactive agent in a cannabinoid sample, comprising:
obtaining the cannabinoid sample in the form of a lipid solution, the cannabinoid sample comprising a first concentration of a psychoactive agent and at least one non-psychoactive agent;
exposing the cannabinoid sample to an effective amount of an oxidizing agent to oxidize at least a fraction of the psychoactive agent;
combining the cannabinoid sample with a non-polar solvent to produce a miscible combination; and
separating an organic phase from the miscible combination to produce a cannabinoid product, the cannabinoid product having a second concentration of the psychoactive agent lower than the first concentration of the psychoactive agent.

2. The method of claim 1, wherein the second concentration of the psychoactive agent is less than 1%.

3. The method of claim 1, wherein the cannabinoid sample comprises or was produced from a hemp extract.

4. The method of claim 1, wherein the cannabinoid sample comprises or was produced from a biosynthesized composition.

5. The method of claim 1, further comprising controlling temperature and/or pressure of the cannabinoid sample.

6. The method of claim 1, wherein separating the organic phase from the miscible combination comprises at least one of a phase separation process and a distillation process.

7. The method of claim 1, wherein the psychoactive agent is tetrahydrocannabinol (THC).

8. The method of claim 7, wherein the THC comprises delta-8 THC, delta-9 THC, delta-10 THC, THCa, THCv, THCp, or a combination thereof.

9. The method of claim 1, wherein the non-psychoactive agent comprises cannabidiol (CBD), cannabinol (CBN), cannabigerol (CBG), cannabichromene (CBC), cannabidivarin (CBDV), or a combination thereof.

10. The method of claim 1, wherein the oxidizing agent comprises ultraviolet (UV) light, air, oxygen, ozone, peroxide, a halogen, potassium nitrate, nitric acid, or a combination thereof.

11. The method of claim 1, wherein the non-polar solvent comprises an alkane, an aromatic, supercritical CO₂, acetic acid, chloroform, diethyl ether, ethyl acetate, methylene chloride, pyridine, ethanol, methanol, or a combination thereof.

12. The method of claim 1, further comprising:
obtaining a cannabinoid starting material in the form of a non-lipid starting material;
distilling the cannabinoid starting material to obtain a cannabinoid distillate;
combining the cannabinoid distillate with an aqueous based oil solution to obtain a cannabinoid lipid mixture; and
separating a lipid phase from the cannabinoid lipid mixture to produce the cannabinoid sample in the form of the lipid solution.

13. A method of reducing THC in a cannabinoid sample having more than 1% THC, the method comprising:
exposing the cannabinoid sample to an effective amount of an oxidizing agent to oxidize a predetermined concentration of the THC and produce an intermediate product;
measuring a concentration of THC in the intermediate product;
responsive to the concentration of THC in the intermediate product being below a predetermined threshold, quenching the intermediate product to produce a cannabinoid product having less than 1% THC.

14. The method of claim 13, further comprising:
responsive to the concentration of THC in the intermediate product being above the predetermined threshold, continuing exposure of the intermediate product to the oxidizing agent.

15. The method of claim 13, further comprising separating an organic phase from the intermediate product to produce the cannabinoid product.

16. The method of claim 13, further comprising measuring the concentration of THC in the cannabinoid sample.

17. The method of claim 16, further comprising selecting the predetermined concentration of THC to be oxidized responsive to the measured concentration of THC in the cannabinoid sample.

18. A system for reducing concentration of a psychoactive agent in a cannabinoid sample, the system comprising:
a reactor having an inlet fluidly connectable to a source of the cannabinoid sample, a test sample outlet, an intermediate product outlet, and a waste outlet, the reactor comprising an insulation layer;
a distillation column fluidly connected to the intermediate product outlet, the distillation column having a residue outlet fluidly connected to a recycle line connectable back to the distillation column and a cannabinoid product outlet;
a temperature sensor positioned to measure temperature within the reactor;
a pressure sensor positioned to measure pressure within the reactor;
a source of an oxidizing agent fluidly connected to the reactor;
a source of a non-polar solvent fluidly connected to the reactor;
a source of a separation agent fluidly connected to the reactor; and
at least valve positioned to control flow of at least one of the oxidizing agent, the non-polar solvent, and the separation agent into the reactor.

19. The system of claim 18, further comprising a controller operably connected to the at least one valve, the controller programmed to actuate the at least one valve to control flow of the at least one of the oxidizing agent, the non-polar solvent, and the separation agent into the reactor.

20. The system of claim 18, further comprising a controller operably connected to the temperature sensor and the pressure sensor, the controller programmed to control temperature and pressure within the reactor responsive to a measurement received from the temperature sensor or the pressure sensor.
